(19) United States
(12) Patent Application Publication
(10) Pub. No.: US 2024/0286567 A1
(43) Pub. Date: Aug. 29, 2024

(54) METHOD FOR CREATING AN INDIVIDUAL USER PROFILE IN THE EVENT OF AN OCCURRENCE OF MOTION SICKNESS OF A USER, COMPUTER PROGRAM PRODUCT, AND SUPPORT SYSTEM

(71) Applicant: CARIAD SE, Wolfsburg (DE)

(72) Inventors: Stefan MAYER, Wolfsburg (DE); Sebastian HANRIEDER, Ingolstadt (DE); Markus MUELLER, Ingolstadt (DE); Tobias SCHLEICHER, Ingolstadt (DE); Mareike GRUND, Wolfsburg (DE)

(21) Appl. No.: 18/587,822

(22) Filed: Feb. 26, 2024

(30) Foreign Application Priority Data
Feb. 27, 2023 (DE) 102023104756.6

(51) Int. Cl. B60R 16/037; A61M 21/00; B60W 40/08; G06V 40/16; G06V 40/18; G06V 40/20; H04L 67/306

(52) U.S. Cl. CPC B60R 16/037; A61M 21/00; B60W 40/08; G06V 40/16; G06V 40/18; G06V 40/20; H04L 67/306; B60W 2040/0872

(57) ABSTRACT

The disclosure relates to a method for creating an individual user profile in an event of an occurrence of motion sickness, including: providing an initial user profile for a user of a motor vehicle; capturing at least one characteristic currently characterizing the user of the motor vehicle; generating a user query; outputting the user query at an output device; capturing an input of the user of the motor vehicle based on the user query; linking the input of the user of the motor vehicle to the at least one characteristic currently characterizing the user of the motor vehicle; and creating the individual user profile by adapting the initial user profile based on the input of the user of the motor vehicle linked to the at least one characteristic currently characterizing the user of the motor vehicle. Furthermore, the disclosure relates to a computer program product, and a support system.

### BACKGROUND

### Technical Field

The disclosure relates to a method for creating an individual user profile in the event of an occurrence of motion sickness of a user of a motor vehicle by way of a support system of the motor vehicle. Furthermore, the disclosure relates to a computer program product, and a support system.

### Description of the Related Art

Systems are already known from the prior art, which, upon occurrence of motion sickness of a user in the motor vehicle, can initiate appropriate countermeasures. However, these countermeasures are very general and not tailored to the user, so that, accordingly, for example, countermeasures are initiated even though the user does not experience any motion sickness.

DE 10 2017 223 609 A1 describes a system for reducing kinetosis for a vehicle, wherein the system has a control unit, which is coupled to a sensor system and/or a navigation system, a vehicle seating system and/or a display unit for receiving and/or sending signals. The control unit is set up, depending on received environmental data and/or vehicle component data, to generate seat adjustment signals and/or display signals.

DE 10 2020 212 434 A1 relates to a device for preventing or reducing kinetosis of at least one vehicle occupant in a vehicle, wherein the device has a determination unit for determining ride-impacting route parameters for a selected route with a starting point and a destination point, wherein the ride-impacting route parameters comprise at least the significant parameters in relation to route planning, has a diagnostic sensor unit, which is designed to identify a current kinetosis value of the at least one vehicle occupant during the ride. The disclosure further relates to a method for preventing or reducing kinetosis, and a route planner, an estimation unit being provided, which is designed to estimate a final kinetosis value of the at least one vehicle occupant, assuming completion of the selected route, at least using the current kinetosis value and the remaining determined route parameters for the selected route with the current location as the starting point.

DE 11 2020 000 867 T5 describes a motion sickness estimation device, including: a sensory conflict amount calculating unit for calculating a conflict amount, which is a conflict amount between a plurality of types of sensory amounts perceived by an occupant related to a motion of the occupant's head, on the basis of the motion of the occupant's head caused by vibration of a vehicle; a traveling situation feature extracting unit for extracting a feature of a traveling situation in relation to the motion sickness from the travel situation on the basis of at least one of the motion of the occupant's head or a motion of the vehicle; a habituation progress state determining unit for determining whether or not the occupant's habituation to the traveling situation has progressed on the basis of biometric information of the occupant; a sensitivity setting unit for setting sensitivity to the feature of the traveling situation on the basis of the habituation progress state; a sensory conflict amount correcting unit for correcting the sensory conflict amount on the basis of the sensitivity; and a sickness estimating unit for estimating a motion sickness state of the occupant on the basis of the sensory conflict amount that has been corrected.

### BRIEF SUMMARY

It is the object of the present disclosure to create a method, a computer program product, and a support system by way of which motion sickness of an individual user can be detected and, in particular, which can be used to respond appropriately to said motion sickness.

One aspect of the disclosure relates to a method for creating an individual user profile in the event of an occurrence of motion sickness of a user of a motor vehicle by way of a support system of the motor vehicle. An initial user profile for users is provided by way of an electronic computing device of the support system. At least one characteristic currently characterizing the user is captured by way of a capture device of the support system. A user query is generated by way of the electronic computing device. The user query is output at an output device of the support system. An input of the user depending on the user query is captured by way of an input device of the support system. The input is linked to the currently captured characterizing characteristic by way of the electronic computing device, and the individual user profile is created by adapting the initial user profile depending on the link by way of the electronic computing device.

This means that motion sickness can occur reliably and individually detected in a user of the motor vehicle. The user of the motor vehicle can be both a driver and a passenger of the motor vehicle. In particular, motion sickness, which is also known as kinetosis, is a highly individual phenomenon. This applies to both susceptibility in general and to the particularities, particularly what type of symptoms are exhibited by the user, in particular. The idea is to gradually determine individual measures of motion sickness/kinetosis based on several factors that can be determined by appropriate sensors or capture devices in the motor vehicle.

As already mentioned, the parameter values vary from operator to operator or from user to user. At the individual level, however, a combination of these parameters can be a characteristic indicator, which can be captured and evaluated via the user profile.

If, for example, the measurement indicates, that a user or an occupant suffers from motion sickness, the motor vehicle can confirm this by having a dialog with the user by way of a digital assistant, for example, via the output device. This could be done, for example, by low-threshold questions such as “How are you feeling.” If the answers or inputs of the user confirm the suspicion of impending nausea, this can serve to reinforce the parameters set for this user.

In addition to the preselected set of parameters, which in particular is provided via the initial user profile and thus potential indicators are provided, it is now possible to observe new correlations or links to motion sickness. This way that the user profile can be created very individually and motion sickness can be detected individually.

The adapted user profile or the individual user profile can be stored in the motor vehicle and also used for future rides and, for example, then provided as an initial user profile. Furthermore, alternatively or additionally, the user profile can be stored on a central electronic computing device, e.g., a cloud server, and be downloaded in another motor vehicle after the user has been actively identified, for example. This makes it possible for the user profile to be used on different motor vehicles.

According to an advantageous embodiment at least one current characteristic of a user's eye is captured as the currently characterizing characteristic by way of an interior camera. The camera can also be designed as an infrared camera in this case. For example, an increased blinking frequency or squinting eyes can be identified by the interior camera, which indicates that the user is straining his/her eyes. Furthermore, an increased blinking frequency or squinting eyes can also indicate difficulties to focus, both of which are symptoms of motion sickness in particular.

Furthermore, it has proven to be advantageous, if at least one current characteristic of a mouth and/or neck of the user are/is captures as the current characterizing characteristic by way of an interior camera. For example, an increase in swallowing processes can be captured, which can be captured, for example, by way of the interior camera via the motion of the larynx, whereby the increase in swallowing processes can also be indicative of motion sickness.

Furthermore, it has proven to be advantageous if, for example, a current sweat production of the user is captured as the currently characterizing characteristic by way of a, for example, optical capture device, for example, a camera or a temperature detection device or by way of a skin conductance determination. For example, sweat production can be captured on a forehead of the user. In particular, sweat can be captured based on captured reflections on the forehead, for example. Increased sweating can also be regarded as a symptom of motion sickness.

A further advantageous embodiment provides that a current motion of an upper body of a user is captured as the current characterizing characteristic by way of a, for example, optical capture device, for example, by way of a camera or also by way of a temperature capture device. In particular, characteristic motions of the upper body and of the larynx can also be indicative that a corresponding motion sickness will set in in the future or has already set in.

Another advantageous embodiment provides, that motion sickness is detected depending on the user profile and/or a countermeasure is initiated against a detected motion sickness. For example, in case of a detected motion sickness it can be provided that an air conditioning device is controlled accordingly, and, for example, the temperature in the inner space is reduced. Furthermore, for example, the supply of fresh air can be initiated, for example via appropriate ventilation systems or by opening the window. This way that motion sickness can be detected early and individually, as well as appropriate countermeasures can be initiated.

A further advantageous embodiment provides that the user continues to be observed during the countermeasure and the countermeasure is evaluated with regard to individual effectiveness, the evaluation being taken into account when adapting the individual user profile. Just like the occurrence of motion sickness itself, the effectiveness of appropriate countermeasures is also highly individual. In particular, the user can be observed during the countermeasure initiated and it can be checked whether the countermeasure has a corresponding effect. For example, for a user, opening the window can be effective, while merely reducing the interior temperature, for example, has no effect. This can now be observed and analyzed and also stored in the user profile. For this purpose, it can also be provided that, for example, a user query is carried out as to whether the corresponding countermeasure resulted in an improvement/a reduction of motion sickness. This is stored and can be used in the future to prevent or treat motion sickness in an automated and improved manner.

It is also advantageous if the electronic computing device is provided with artificial intelligence. For example, artificial intelligence can be provided as a neural network. In particular, this is a learning electronic computing device in which both the user profile can be adapted accordingly with regard to the occurrence of motion sickness and appropriate countermeasures can also be learned. Thus, motion sickness can be detected or prevented most individually and reliably.

The method presented is in particular a computer-implemented method. Therefore, a further aspect of the disclosure relates to a computer program product with program code way, which cause an electronic computing device to carry out a method according to the previous aspect when the program code is processed by the electronic computing device.

A further aspect of the disclosure therefore also relates to a computer-readable storage medium including the computer program product according to the previous aspect.

The disclosure also relates to a support system for a motor vehicle for creating an individual user profile in the event of the occurrence of motion sickness of a user of the motor vehicle, including at least a capture device, an output device, an input device and an electronic computing device, the support system being designed for carrying out a method according to the previous aspect. In particular, the method is carried out by way of the support system.

The electronic computing device has, for example, processors, circuits, especially integrated circuits, as well as more electronic components in order to be able to carry out the corresponding acts of the method.

Furthermore, the disclosure also relates to a motor vehicle with a support system according to the previous aspect. Here, the motor vehicle can be designed both as an at least partially autonomous motor vehicle and as a fully autonomous motor vehicle. The support system is also designed as a manually driven motor vehicle.

Advantageous embodiments of the method are to be regarded as advantageous embodiments of the computer program product as well as the support system and the motor vehicle. For this purpose, the support system as well as the motor vehicle have relevant features in order to be able to carry out the corresponding steps of the method.

For specific applications or application situations that may arise when carrying out the method and which are not explicitly described here, it can be provided that according to the method an error message and or a prompt for inputting user feedback is output, and/or a default setting and or a predetermined initial state is set.

The disclosure also includes the control device/electronic computing device for the support system. The control device can have one data processing device or a processor device, which is set up to carry out an embodiment of the method according to the disclosure. For this purpose, the processor device can have at least a microprocessor and/or at least a microcontroller and/or at least a FPGA (Field Programmable Gate Array) and/or at least a DSP (Digital Signal Processor). Furthermore, the processor device can have program code that is set up, when executed by the processor device, to carry out the embodiment of the method according to the disclosure. The program code can be stored in a data memory of the processor device. The processor circuit of the processor device can, for example, have at least one circuit board and/or at least a SoC (System on Chip).

The disclosure also includes refinements of the support system according to the disclosure, which have features such as those already described in the context with the refinements of the method according to the disclosure. For this reason, the corresponding refinements of the traffic control system according to the disclosure are not described again here.

As a further solution, the disclosure also comprises a computer-readable storage medium, comprising program code that, when executed by a processor circuit of a computer or a computer network, causes it to carry out an embodiment of the method according to the disclosure. The storage medium can, for example, be provided at least partially as a non-volatile data memory (e.g., as a flash memory and/or as an SSD-Solid State Drive) and/or at least partially as a volatile data memory (e.g., as RAM-Random Access Memory). The storage medium can be arranged in the data memory of the processor circuit. The storage medium can also be operated, for example, as a so-called app store server in the Internet. A processor circuit with at least one microprocessor can be provided by the computer or computer network. The program code can be provided as binary code or assembler and/or as source code of a programming language (e.g., C) and/or as a program script (e.g., Python).

The disclosure also comprises the combinations of the features of the described embodiments. The disclosure therefore also comprises implementations that each have a combination of the features of several of the described embodiments, provided the embodiments have not been described as being mutually exclusive.

### BRIEF DESCRIPTION OF THE SEVERAL VIEWS OF THE DRAWINGS

Below, exemplary embodiments the disclosure are described. In the drawings:

FIG. 1 shows a schematic side view of an embodiment of a motor vehicle with an embodiment of a support system; and

FIG. 2 shows a schematic flow diagram according to an embodiment of a method.

### DETAILED DESCRIPTION

The exemplary embodiments explained below are preferred embodiments of the disclosure. In the exemplary embodiments each of the described components of the embodiments represent individual, independent features of the disclosure, which also refine the disclosure independently. Therefore, the disclosure is intended to comprise combinations of the features of the embodiments other than those shown. Furthermore, the described embodiments can also be supplemented by further features of the disclosure that have already been described.

In the Figures, same reference numerals designate functionally identical elements in each case.

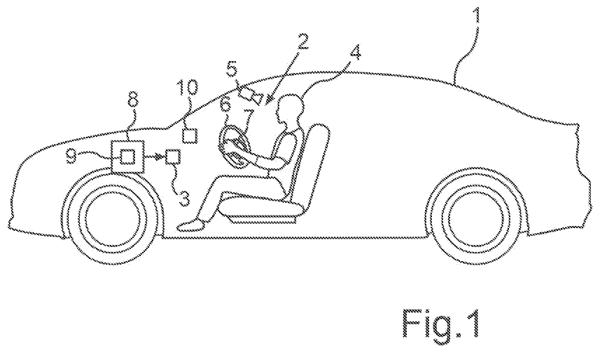

FIG. 1 shows a schematic side view of an embodiment of a motor vehicle 1. Motor vehicle 1 is pure exemplary designed as at least partially autonomous motor vehicle 1 or fully autonomous motor vehicle 1. Motor vehicle 1 can also be designed as a motor vehicle 1 that can merely be operated by a driver. Motor vehicle 1 has a support system 2. Support system 2 is designed for creating an individual user profile 3 in the event of an occurrence of motion sickness of a user 4 of motor vehicle 1. Here, user 4 is shown in a front area of motor vehicle 1. User 4 can sit as a driver, as a passenger or, as shown, in a rear area.

Support system 2 has at least a capture device 5, an output device 6, an input device 7, and an electronic computing device 8. In the following exemplary embodiment, input device 7 and output device 6 are designed, for example, via a touch-sensitive display. For example, an input by user 4 can be made at the display and information from electronic computing device 8 can be displayed on the display. Capture device 5 can be designed, for example, as a camera, in particular as an interior camera. The camera can also be designed as an infrared camera. Furthermore, capture device 5 can also be designed as a temperature capture device or the like.

Electronic computing device 8 has in particular artificial intelligence 9, for example, in the form of a neural network. Furthermore, a device 10 for carrying out a countermeasure against any detected motion sickness is shown. For example, device 10 can be regarded as an air conditioning device or as a window or as a seat adjustment device of motor vehicle 1.

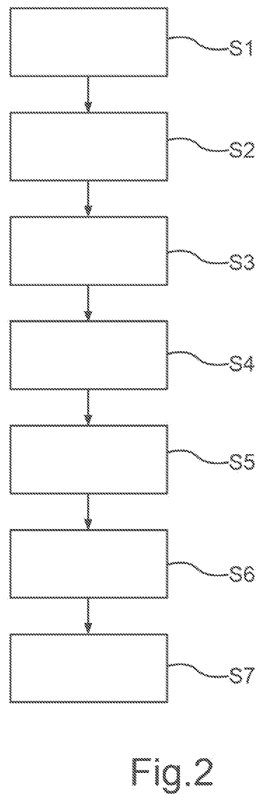

FIG. 2 shows a schematic flow diagram according to an embodiment of the method. In particular, FIG. 2 shows a method for creating individual user profile 3 in the event of an occurrence of motion sickness of a user 4 of motor vehicle 1 by way of support system 2. In a first step S1, an initial user profile for users is provided by way of an electronic computing device 8. In a second step S2, at least one characteristic that currently characterizes user 4 is captured by way of capture device 5. In the third step S3, a user query is generated by way of electronic computing device 8. In a fourth step S4, the user query is output at output device 6. In a fifth step S5, an input of user 4 is captured depending on the user query by way of input device 7. In a sixth step S6, the input is linked to the currently captured characterizing characteristic by way of electronic computing device 8. In a seventh step S7, the individual user profile 3 is again created by adapting the initial user profile depending on the link by way of electronic computing device 8.

In particular, it can be provided, that at least a current characteristic of an eye of user 4 is captured as the currently characterizing characteristic by way of the interior camera. For example, strained eyes or difficulties focusing can be captured accordingly. Especially, an increased tendency to blink or squinting eyes can suggest in turn strained eyes as well as difficulties focusing.

Furthermore, it can be provided, that at least a current characteristic of a mouth and/or a neck of user 4 is captured as the current characterizing characteristic. For this purpose, it can be provided, for example, that the increase in swallowing processes can be captured, which can be determined in particular via the motions of the larynx of user 4.

Furthermore, it can be provided that a current sweat production of user 4 is captured as the currently characterizing characteristic by way of an optical capture device, for example, by way of the interior camera or also by way of a temperature capture device. In particular, reflections on the forehead, for example, can be used to determine corresponding sweat production. Furthermore, it can also be provided that corresponding motions of the upper body of user 4 and also of the larynx can be captured by capture device 5, in particular by an optical capture device.

It can furthermore be provided that motion sickness is detected depending on the user profile 3 and/or a countermeasure, for example via device 10, is initiated against detected motion sickness. In this case it can be provided further that user 4 is observed further during the countermeasure, and the countermeasure is evaluated with regard to individual effectiveness, the evaluation being taken into account when adapting user profile 3.

In particular, it thus can be provided, that user profile 3 can be adapted accordingly, because the parameter values of users 4 can vary greatly and therefore, on an individual level, a combination of these parameters can be a characteristic indicator of motion sickness. If the corresponding measurements or captures suggest that user 4 is suffering from motion sickness, motor vehicle 1 can confirm this by having a dialog with user 4, for example, via support system 2. This can be done by low-threshold questions such as, for example, “How are you feeling?” If the answers of user 4 confirm the suspicion of impending nausea, this can serve to reinforce the parameters set for this user.

In addition to the preselected set of parameters and as potential indicators for the motion sickness, which corresponds in particular to the initial user profile, it is now possible to observe new correlations to motion sickness. For example, by evaluating the entire sensor output via an artificial intelligence 9, for example a neural network, new individual symptoms such as turning down the air conditioning or body motions in the seat can be determined accordingly. In particular, a corresponding pressure device can also be provided in the seats as a capture device, which can record appropriate motions.

By combining the results of all users, in turn, a general model of the motion sickness can be derived, that can be used as basis for individual models, thus, in particular, the initial user profile.

On the basis of the individual model of the motion sickness, in turn, countermeasures against motion sickness can be initiated. These countermeasures can also be adapted to individual users 4 and their activities. For example, when watching a movie, opening the window for fresh air will probably be a less accepted solution than lowering the air conditioning temperature. Support system 2 of motor vehicle 1 can also contribute indirectly to treat motion sickness, for example by support system 2 proactively pointing out points of interest in the front area of motor vehicle 1 and prompt user 4 to look ahead. For example, for this purpose, corresponding points of interest can be used, which draw user's 4 attention to interesting circumstances in the environment in a particularly individual way for user 4. In order to evaluate the effectiveness of the countermeasures in a low-threshold dialog with the digital assistants or support system 2 of motor vehicle 1, the corresponding activities or symptom parameters of user 4 can also be evaluated accordingly while the countermeasure is being carried out.

German patent application no. 10 2023 104 756.6, filed Feb. 27, 2023, to which this application claims priority, is hereby incorporated herein by reference, in its entirety.

Aspects of the various embodiments described above can be combined to provide further embodiments. In general, in the following claims, the terms used should not be construed to limit the claims to the specific embodiments disclosed in the specification and the claims, but should be construed to include all possible embodiments along with the full scope of equivalents to which such claims are entitled.

1. A method for creating an individual user profile in an event of an occurrence of motion sickness of a user of a motor vehicle by way of a support system of the motor vehicle, the method comprising:
providing an initial user profile for the user of the motor vehicle by way of an electronic computing device of the support system;
capturing at least one characteristic that currently characterizes the user of the motor vehicle by way of a capture device of the support system;
generating a user query by way of the electronic computing device;
outputting the user query at an output device of the support system;
capturing an input of the user of the motor vehicle based on the user query by way of an input device of the support system;
linking the input of the user of the motor vehicle to the at least one characteristic that currently characterizes the user of the motor vehicle by way of the electronic computing device; and
creating the individual user profile by adapting the initial user profile based on the input of the user of the motor vehicle linked to the at least one characteristic that currently characterizes the user of the motor vehicle by way of the electronic computing device.

2. The method according to claim 1, wherein
at least one current characteristic of an eye of the user of the motor vehicle is captured as the at least one characteristic that currently characterizes the user by way of an interior camera.

3. The method according to claim 1, wherein
at least one current characteristic of a mouth and/or a neck of the user of the motor vehicle is captured as the at least one characteristic that currently characterizes the user of the motor vehicle by way of an interior camera.

4. The method according to claim 1, wherein
a current sweat production of the user of the motor vehicle is captured as the at least one characteristic that currently characterizes the user of the motor vehicle by way of the capture device.

5. The method according to claim 1, wherein
a current motion of an upper body of the user of the motor vehicle is captured as the at least one characteristic that currently characterizes the user of the motor vehicle by way of the capture device.

6. The method according to claim 1, further comprising:
detecting motion sickness based on the individual user profile and/or initiating a countermeasure against detected motion sickness.

7. The method according to claim 6, further comprising:
observing the user of the motor vehicle during the countermeasure; and
evaluating the countermeasure with regard to individual effectiveness, wherein the adapting the initial user profile takes into account the evaluating.

8. The method according to claim 1, wherein
the electronic computing device is provided with artificial intelligence.

9. A non-transitory computer-readable medium storing a computer program with program code that causes an electronic computing device to:
provide an initial user profile for a user of a motor vehicle;
capture at least one characteristic that currently characterizes the user of the motor vehicle by way of a capture device;
generate a user query;
output the user query at an output device;
capture an input of the user of the motor vehicle based on the user query by way of an input device;
link the input of the user of the motor vehicle to the at least one characteristic that currently characterizes the user of the motor vehicle; and
adapt the initial user profile based on the input of the user of the motor vehicle linked to the at least one characteristic that currently characterizes the user of the motor vehicle to create an individual user profile.

10. A support system that creates an individual user profile in an event of an occurrence of motion sickness of a user of a motor vehicle, the support system comprising:
a capture device;
an output device;
an input device; and
an electronic computing device that, in operation:
provides an initial user profile for the user of the motor vehicle;
captures at least one characteristic that currently characterizes the user of the motor vehicle by way of the capture device;
generates a user query;
outputs the user query at the output device;
captures an input of the user of the motor vehicle based on the user query by way of the input device;
links the input of the user of the motor vehicle to the at least one characteristic that currently characterizes the user of the motor vehicle; and
adapts the initial user profile based on the input of the user of the motor vehicle linked to the at least one characteristic that currently characterizes the user of the motor vehicle to create an individual user profile.
